(19) United States
(12) Patent Application Publication
(10) Pub. No.: US 2010/0113821 A1
(43) Pub. Date: May 6, 2010

(54) PREPARATION OF 3-AMINO-3-(CYCLOBUTYLMETHYL)-2-(HYDROXY)-PROPIONAMIDE HYDROCHLORIDE

(75) Inventors: Jeonghan Park, Whippany, NJ (US); Eugene J. Vater, Lyndhurst, NJ (US); Shuan Dong, Watchung, NJ (US); Tetsuo Iwama, Scotch Plains, NJ (US); Ramani R. Raghavan, Lexington, MA (US); Hong-Chang Lee, Livingston, NJ (US); George S.k. Wong, Summit, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering-Plough Corporation, Kenilworth, NJ (US)

(21) Appl. No.: 12/519,486

(22) PCT Filed: Dec. 17, 2007
(86) PCT No.: PCT/US07/25759

§ 371 Date: Jan. 8, 2010

Related U.S. Application Data
(60) Provisional application No. 60/876,028, filed on Dec. 19, 2006.

(51) Int. Cl. C07C 69/74; C07C 229/02; C07C 237/14; C07C 205/15; C07C 205/02

(52) U.S. Cl. 560/123; 562/505; 564/191; 568/704; 568/924

(57) ABSTRACT

Disclosed is a process for preparing 3-(amino)-3-cyclobutylmethyl-2-hydroxy-propionamide hydrochloride, an intermediate useful in the preparation of the HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide.

### FIELD OF THE INVENTION

The present invention relates to a process for the preparation of 3-(amino)-3-cyclobutylmethyl-2-hydroxy-propionamide hydrochloride, an intermediate useful in the preparation of the HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide.

### BACKGROUND

Hepatitis C virus (HCV) is a (+)-sense single-stranded RNA virus that has been implicated as the major causative agent in non-A, non-B hepatitis; an HCV protease necessary for polypeptide processing and viral replication has been identified. U.S. Pat. No. 7,012,066 discloses a genus of HCV protease inhibitors that includes (1R,5S)—N-[3-amino-1-(cyclobutylmethyl)-2,3-dioxopropyl]-3-[2(S)-[[[(1,1-dimethylethyl)amino]-carbonyl]amino]-3,3-dimethyl-1-oxobutyl]-6,6-dimethyl-3-azabicyclo[3.1.0]hexan-2(S)-carboxamide, having the structural formula

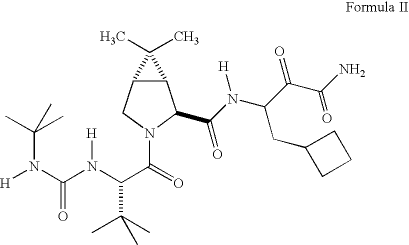

US2005/0059800, published Mar. 17, 2005, and U.S. Provisional Application No. 60/876,447, filed Dec. 20, 2007, each disclose processes for preparing the compound of Formula II, each of which is incorporated herein by reference in its entirety. Additionally, published U.S. Patent Application No. 2007/0149459, filed Nov. 13, 2006, discloses oxidation processes for preparing the compound of Formula II. Methods for preparing diastereomers of the compound of Formula II are disclosed in Published U.S. Patent Application No. 2005/0249702, filed Nov. 10, 2005.

Published U.S. Patent Application No. 2005/0020689, filed Jan. 27, 2005, discloses processes for preparing 3-(amino)-3-cyclobutylmethyl-2-hydroxy-propionamide hydrochloride from (diphenylmethylene)glycine ethyl ester.

In general, the compound of Formula II is prepared in accordance with the process of Scheme I,

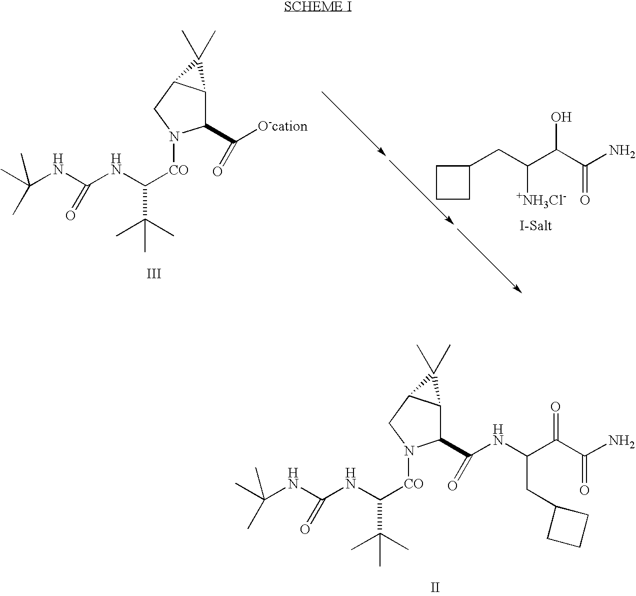

wherein the compound of Formula III is coupled to a salt (I-Salt) of the compound of Formula I. With reference to Scheme I, what is needed is an improved method for the provision of intermediate compound of Formula I.

### SUMMARY OF THE INVENTION

In one aspect, the present invention comprises a process for preparing 3-(amino)-3-cyclobutylmethyl-2-hydroxy-propionamide hydrochloride, the intermediate compound of Formula I (which represents all diastereomers),

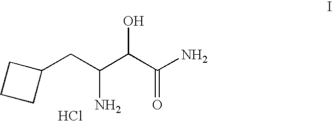

the method comprising:
(A) coupling nitroalkane (E) with glyoxylic acid to obtain the nitro-hydroxy acid (F):

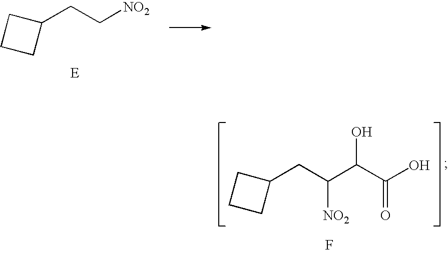

wherein (E) is prepared by a process comprising:
(1) oxidizing cyclobutanemethanol (A) with 2,2,6,6-tetramethyl-1-piperidinyloxy, free radical, to obtain cyclobutanecarboxaldehyde (B):

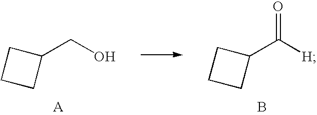

(2) coupling the aldehyde (B) with nitromethane to obtain the nitro-alcohol (C):

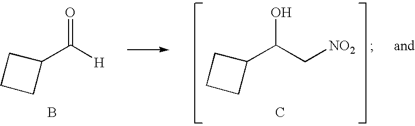

(3) converting compound “C” to compound “E” by
   (a) a first method comprising:
      (i) reacting nitro-alcohol (C) with acetic anhydride to obtain a mixture of compounds (CC) and (D):

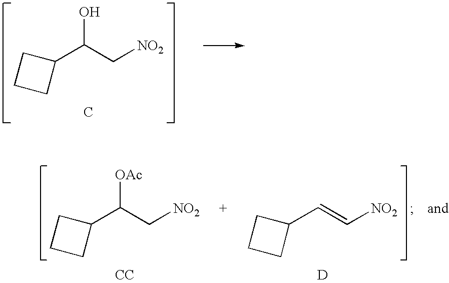

(ii) converting the mixture of (CC) and (D) obtained in Step (a)(i) to nitroalkane (E) by a process selected from:

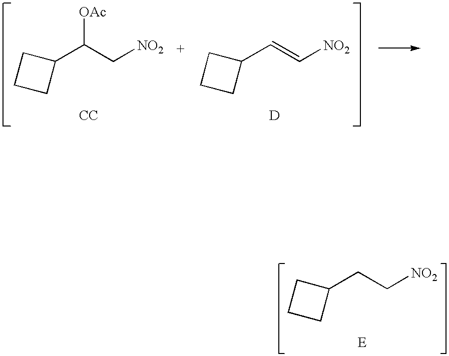

I) hydrogenation of the mixture; or
II) reduction of the mixture with sodium borohydride in the presence of PEG-400; or
III) reduction of the mixture with sodium borohydride in the presence of an alcohol, or
(b) a second method comprising:
   (i) reacting the nitro-alcohol (C) with CH₃SO₂Cl and triethylamine to obtain compound (D):

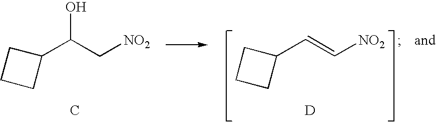

(ii) reducing (D) obtained in second process Step (b)(i), thereby providing nitroalkane (E);
(B) hydrogenating compound (F) provided by Step “A” to yield amino-hydroxy acid (FA):

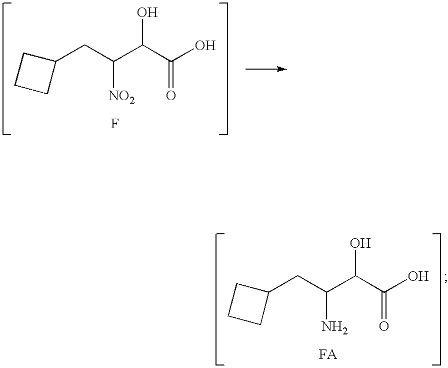

3) refluxing (FA) with p-toluenesulfonic acid and esterifying to obtain (FF):

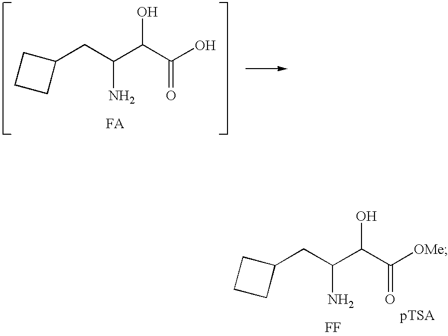

4) converting the ester to an amide and protecting the amino group of (FF) to obtain (G), wherein Prot is a protecting group:

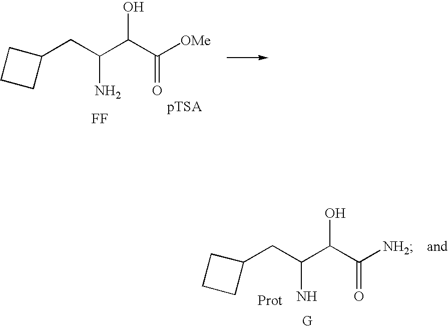

5) heating (G) in a solution of HCl in alcohol.

In another embodiment, a racemic precipitate comprising an isolated pair of enantiomers of the compound of Formula F is prepared by reacting the isolated compound of Formula (E) with glyoxylic acid and triethylamine, followed by benzylamine, to form a benzyl amine salt of a pair of enantiomers of Formula F′-BA, that is, the benzyl amine salt of the RS and SR diastereomers (which are enantiomers) of the compound of Formula F, shown below as the F′ enantiomeric pair.

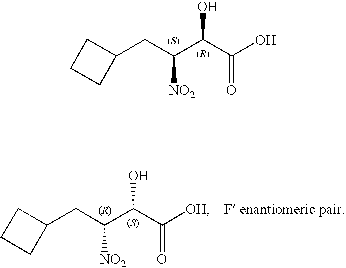

In this embodiment, the benzylamine salt of the enantiomeric pair of compounds of Formula F′-BA precipitates preferentially from a solution comprising all of the diastereomers of the compound of Formula F. In some embodiments it is preferred to employ a sufficient amount of benzyl amine with the racemic mixture of the compound of Formula F to precipitate an amount of the SR and RS form of the salt that exceeds the amount of the SS and RR isomers present in an equilibrium solution of all diastereomers, and to carry out precipitation of the SR and RS isomers under conditions wherein the diasteromers present in the mixture in the SS and RR form are interconverted to an SR or RS form in situ and are subsequently, selectively precipitated as the benzyl amine salt (F′-BA) (dynamic precipitation).

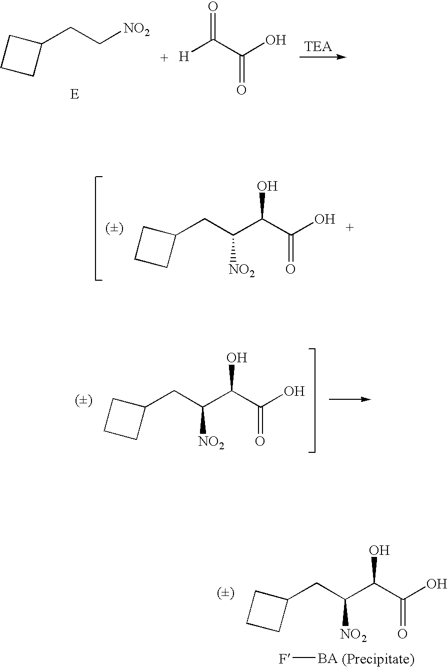

The pair of enantiomers comprising the precipitate of Formula (F′-BA) is acidified and reduced to the corresponding amine (F′A), then esterified to give a pair of enantiomers (F′F) which are converted to the corresponding HCl salt, compound (F′F-HCl):

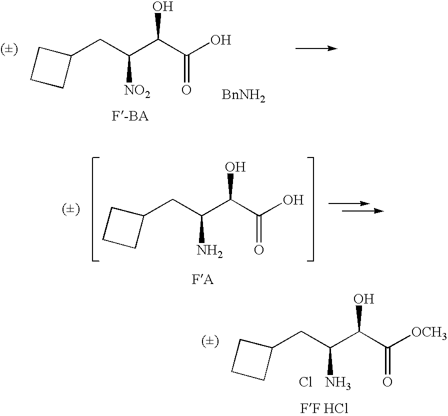

Subsequently, the ester functionality of the precipitated pair of enantiomers (F′F HCl) is converted to the corresponding amide, yielding an amino-hydroxy-amide intermediate, and the nitrogen atom of the amino functional group of the resulting amino-hydroxy-amide is protected to obtain a pair of enantiomers of Formula (G′):

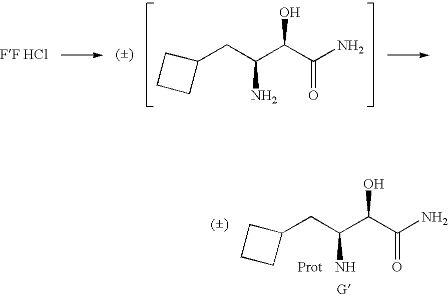

The racemic mixture of the pair of enantiomers of Formula (G′) is heated in an alcoholic HCl solution, deprotecting the amino functional group and yielding the pair of enantiomers of Formula IA, i.e., the enantiomeric compounds having the structure

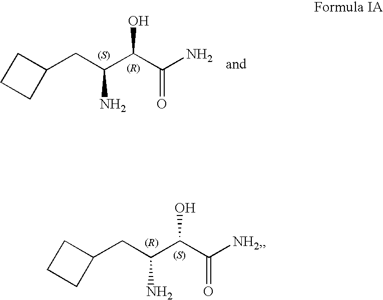

which is sometimes represented herein for convenience as:

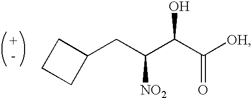

wherein the +/− sign indicates a racemate comprising both the RS and SR enantiomers.

The compound of Formula IA is a useful intermediate in the preparation of the compound of Formula II

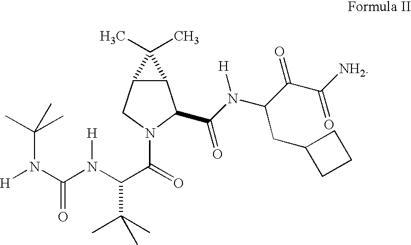

In another aspect of the present invention, dicyclohexylamine (DCHA) is employed in place of benzylamine, providing a precipitate comprising the DCHA salt of the pair of enantiomers of Formula F″, which have the structure:

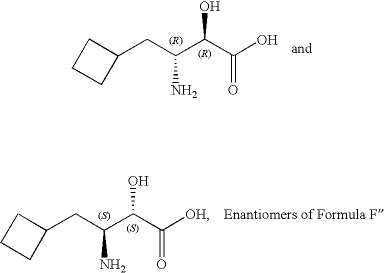

Thus, another aspect of the invention is the provision of the racemic precipitate comprising the pair of enantiomers of Formula F″.

The enantiomers of Formula F″ can be used also in the process described above to prepare a pair of enantiomers comprising the RR and SS forms of the compound of Formula I. As with the enantiomer pair of Formula F′, the enantiomer pair of Formula F″ are also represented herein sometimes for convenience as:

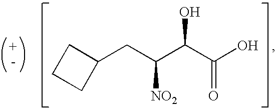

wherein the +/− sign indicates a racemate, that is, the precipitate comprises equal amounts of both the SS and the RR isomers of the compound of Formula I. Accordingly, once isolated, the compound (F″-DCHA) (prepared by treatment of the compounds of Formula F″ with DCHA) is acidified and reduced to the amine (F″A), then esterified and converted to the HCl salt of the ester (F′F):

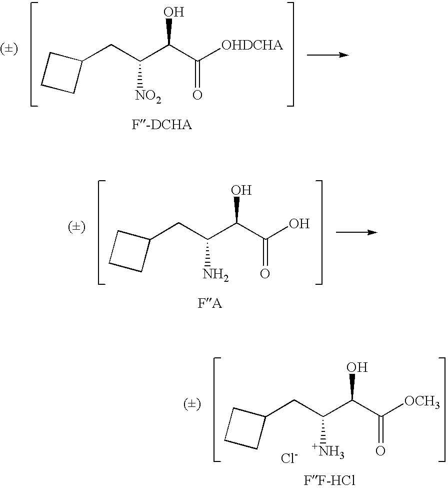

(F″F-HCl) is converted to the amide and the amino group is protected to obtain (G″):

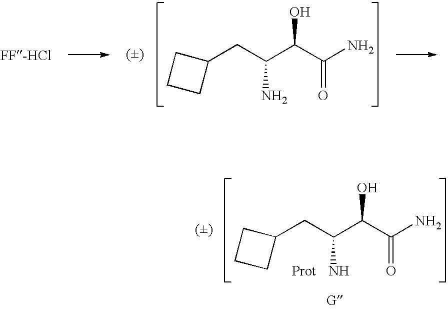

In some embodiments it is preferred to heat the pair of isomers (G″) in an alcoholic HCl solution, deprotecting the amino group, to obtain a pair of diastereomers of Formula IB, i.e., the compounds having the structure

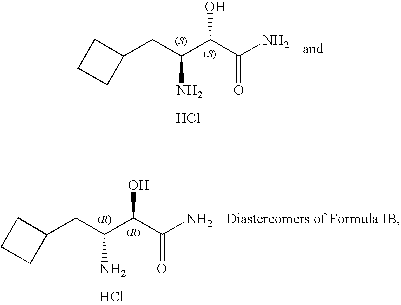

which can be employed also in providing the compound of Formula II.

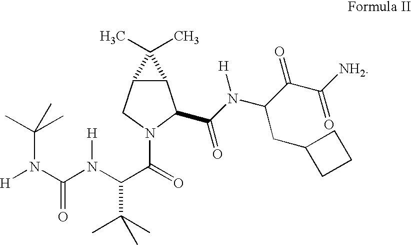

In one aspect, the present invention is the compounds having the following structures and a process for preparing each of those compounds:

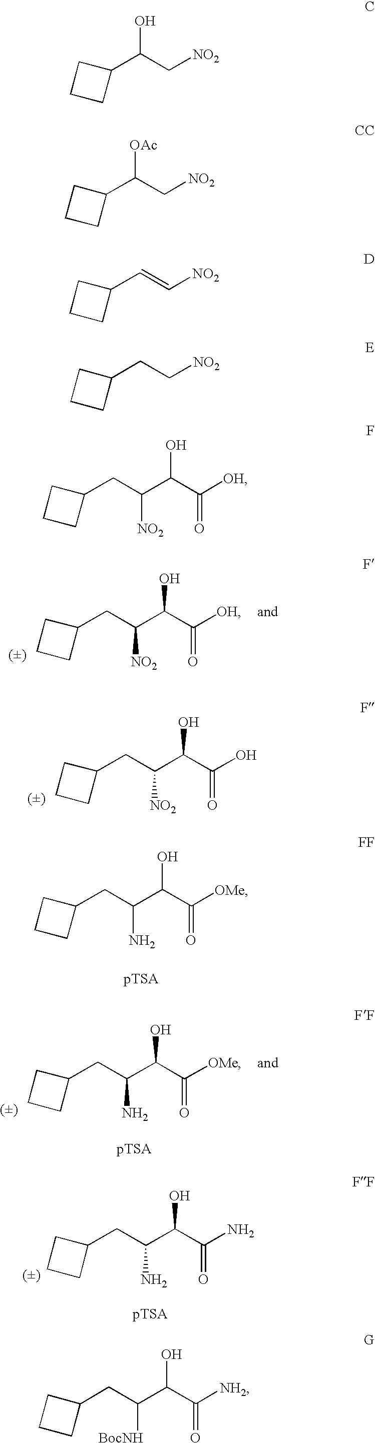

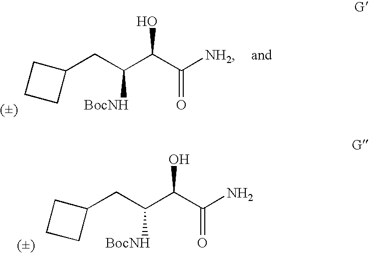

### DETAILED DESCRIPTION

In one aspect, the process of the invention, starting with the preparation of the nitroalkane (E) uses the reaction shown schematically in Scheme 1, below.

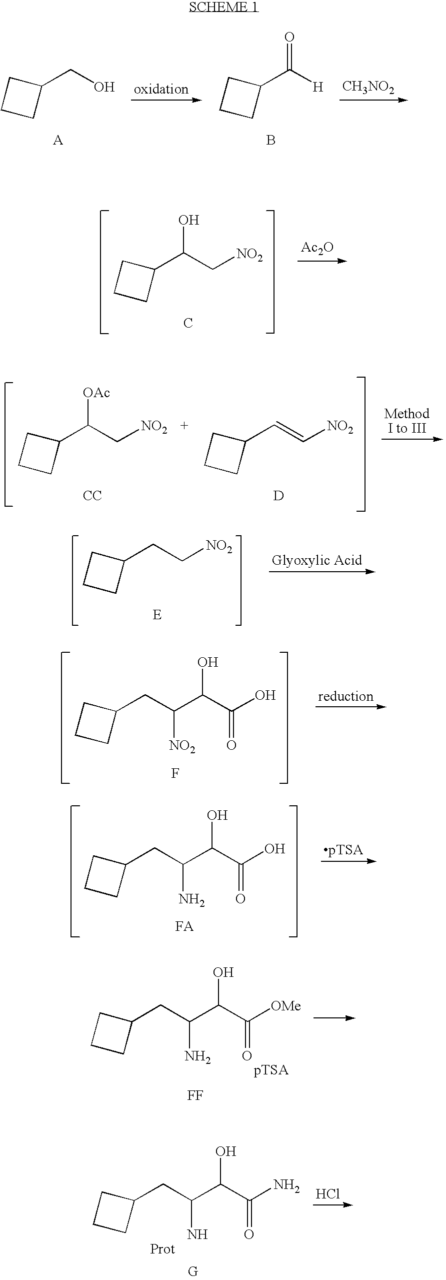

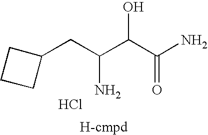

In another aspect, the process of the invention, starting with the preparation of the nitroalkane (E) uses the reaction shown schematically in Scheme 2, below.

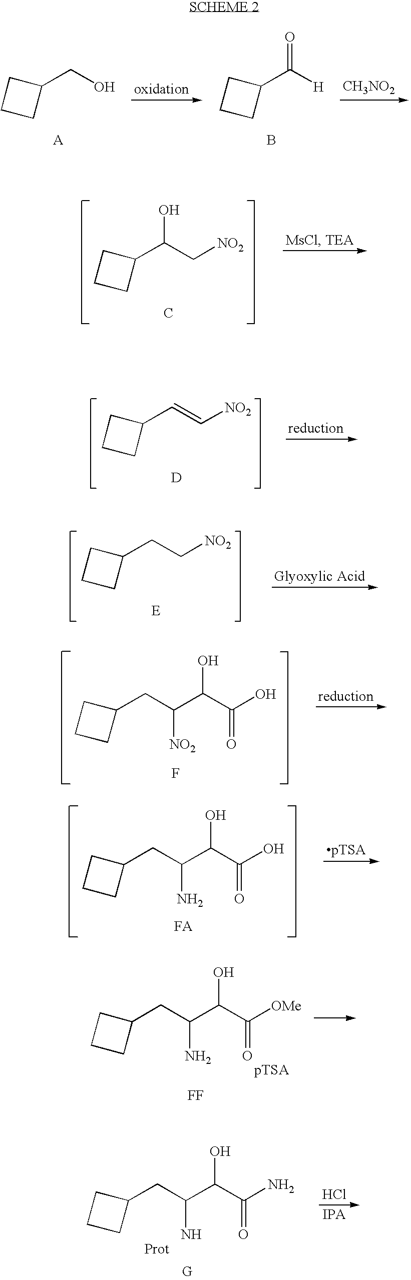

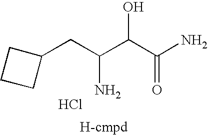

In the Summary of the Invention and in the reaction schemes shown above, the brackets indicate that the intermediates are not isolated before continuing with the next step. It will be appreciated that the intermediate compounds can be isolated, but in some embodiments it is preferred not to isolate the products at each step.

US2005/0020689, which is incorporated herein in its entirety, discloses additional processes for preparing 3-(amino)-3-cyclobutylmethyl-2-hydroxy-propionamide hydrochloride from (diphenylmethylene)-glycine ethyl ester. In addition, procedures for using the intermediate compound(s) of Formula I for preparing the compound of Formula II are disclosed in U.S. Pat. No. 7,012,066. Disclosed in published U.S. Patent Application Nos. 2005/0059800, published ?? and US2005/0249702, published ?? and U.S. application Ser. No. 11/792,770, filed Jun. 8, 2007, and Ser. No. 11/598,528, filed Nov. 13, 2006, each of which is incorporated herein by reference in its entirety, are additional procedures for preparing compounds of Formula II from the intermediate of Formula I.

The following abbreviations are used in the description and examples below: TEMPO (2,2,6,6-Tetramethyl-1-piperidinyloxy, free radical); RT (room temperature); TEA (triethylamine); DMAP (N,N-dimethylaminopyridine); EtOAc (ethyl acetate); IPA (isopropyl alcohol); Ac (acetyl); Et (ethyl); THF (tetrahydrofuran); eq (equivalent(s)); MTBE (tert-butyl methyl ether); Boc (t-butoxy carbonyl). The symbol (±) is inserted in front of a structure having at least one chiral center to indicate that the compound presented structurally along with its enantiomer is present as a racemic mixture, therefore, equal amounts of each enantiomer are present.

Cyclobutanecarboxaldehyde, the compound of Formula (B) is prepared from commercially available cyclobutanemethanol (A) by oxidation, preferably by using the known TEMPO oxidation procedure. The TEMPO reaction is carried out in a solvent such as CH₂Cl₂, EtOAc, toluene or MTBE, preferably CH₂Cl₂ (about 5-15× volume, preferably about 10×), to which is added KBr (about 20-30%, preferably about 24% in water) and NaHCO₃ (preferably a saturated aqueous solution). About 0.005-0.2 eq, preferably about 0.02 eq of TEMPO reagent is added and the mixture cooled to about ±10 to 10° C., preferably about ±5 to 0° C., followed by the addition of 1-1.3 eq, preferably about 1.15 eq, of sodium hypochlorite (bleach). After reaction, KH₂PO₄ or Na₂S₂O₃ is added (about 0.2-0.4 eq, preferably about 0.25 eq), and the product is recovered.

The compound of Formula (B) is converted to the corresponding nitro-alcohol (C) by Henry coupling with nitromethane in the presence of TEA. TEA (about 0.1-1 eq, preferably about 0.3 eq) is added to 1-5 eq, preferably 1.2 eq of nitromethane in a solvent such as toluene, CH₂Cl₂, CH₃OH, ethanol, THF, 2-methyl-THF, ethylene glycol dimethyl ether, MTBE, EtOAc, CH₃CN, isopropyl acetate, or a mixture thereof, preferably toluene. TEA is added and the mixture is agitated. Temperature during the addition is maintained at about 0° C. to about 40° C., preferably about 15° C. to about 25° C., and during the agitation temperature is maintained at a temperature of from about 0° C. to about 40° C., preferably at a temperature of from about 20° C. to about 25° C. The product is preferably used directly in the next step.

Thus prepared, the compound of Formula (C) is converted to a 1:1 mixture of the compounds of Formulae (CC) and (D) by reacting with acetic anhydride in the presence of catalytic amount of DMAP. A catalytic amount of DMAP is added to the solution containing the compound of Formula (C), and about 1-2 eq, preferably about 1.35 eq of acetic anhydride are added. Temperature during the addition is maintained at about 0° C. to about 40° C., preferably about 15° C., and during the agitation temperature is maintained at about 0° C. to about 40° C., preferably at a temperature of from about 15° C. to about 20° C. The product is preferably used directly in the next step.

The solution containing the compounds of Formulae (CC) and (D) prepared above is converted to the corresponding nitro-alkane compound of Formula (E) using one of three different procedures: Method I—hydrogenation of the solution comprising the compounds of Formulae CC and D with hydrogen in the presence of a hydrogenation catalyst; Method II—reduction of the compounds of Formulae CC and D using NaBH₄ in the presence of PEG-400®; and Method III—reduction of the compounds of Formulae CC and D using NaBH₄ in the presence of t-butanol. The resultant crude solution comprising the compound of Formula (E) can be used for the preparation of the compound of Formula (F) in next step, optionally with a distillation step prior to carrying out the conversion to the compound of Formula (F).

When Method I is employed for the conversion of the compounds of Formulae (CC) and (D), to the solution comprising the compounds of Formulae (CC) and (D) is added a solvent, for example, CH₃OH, a base, for example, triethyl amine (TEA) (about 0.1-1 eq, preferably about 0.6 eq), and a catalytic amount of a hydrogenation catalyst, for example, a group 8 metal catalyst, for example, any form of Pd on active charcoal and Ru, preferably Pd/C, and more preferably 5% Pd/C, E101R® from Degussa®. In some embodiments it is preferred to employ hydrogen pressures ranging from about 1-100 psi, more preferably, hydrogen pressures of about 5 psi are preferred. In some embodiments it is preferred to carry out the hydrogenation reaction to obtain the compound of Formula (E) at a temperature of from about (−10)° C. to about (+20)° C., preferably at a temperature of about 0° C.

When Method II is employed for the conversion of the compounds of Formulae (CC) and (D), a solution comprising the compounds of Formulae (CC) and (D) is charged with a polyethylene glycol (PEG) analog, preferably PEG-400, at a range of up to 2 times a number of mL of solvent volume based on the number of grams weight of the starting material present. That is to say that, for example, if 100 g of the compound of Formulae B is used, up to 200 mL of PEG 400 is used. Preferably, a volume in mL of 1 times the number of grams of starting material is used. Solid NaBH₄ is added at a range of 1-4 eq, preferably 2 eq. The addition is carried out at a temperature range of from about (−10)° C. to about (+40)° C., preferably at a temperature of from about (+5)° C. to about (+20)° C. Alternatively, Method II can be carried out by adding the solution of (CC) and (D) to a slurry of about 1-4 eq, preferably about 2.5 eq of NaBH₄ in a solvent, for example, toluene, at a temperature of from about 0° C. to about 40° C., preferably from about 10° C. to about 20° C.

When Method III is employed for the conversion of the compounds of Formulae (CC) and (D) to the compound of Formula (E), a solution comprising the compounds of Formulae (CC) and (D) is charged with an alcohol, for example, t-butanol, isopropyl alcohol (IPA), ethanol (EtOH) and methanol (CH₃OH). In some embodiments it is preferred to employ t-butanol in an amount of a volume in mL of up to 3 times the weight in grams of starting material used, preferably a volume in mL of about 1.89 times the weight in grams of starting material used. For example, if 100 g of the compound of Formulae B is used, up to 300 mL of t-butanol is used. In some embodiments it is preferred to add the alcohol solution of the compounds of Formulae (CC) and (D) to a slurry comprising from about 1 to about 4 equivalents, preferably about 1.5 equivalents, of solid NaBH₄ in a solvent, for example, toluene, at a temperature range of about 0-40° C., preferably about 15-25° C.

To obtain the compound of Formula (F), to the solution of the compound of Formula (E) in toluene is added methanol (about 1-5×, based on the weight of (E), preferably about 3×). Glyoxylic acid, 50% in water, or glyoxylic acid monohydrate is added at a range of about 1-3 eq, preferably about 2 eq, while maintaining the temperature of the reaction mixture at from about 0° C. to about 40° C., preferably at a temperature of from about 0° C. to about 20° C. Triethyl amine (TEA) is added in an amount providing from about 1 equivalent to about 4 eqivalents, preferably about 2.7 equivalents based on the amount of the compound of Formula (E) present, and the mixture agitated while maintaining the temperature at from about 0° C. to about 50° C., preferably about 25° C. to about 35° C. The compound of Formula (F) thus prepared is recovered as a solution in an organic solvent after extraction with base, then acid. In some embodiments it is preferred to employ the solution comprising the compound of Formula (F) in the next step without further purification.

The compound of Formula (F) is converted to the compound of Formula (FA) by adding the solution of Formula (F) obtained in the previous step to an alcohol, for example, methanol, and a catalytic amount, preferably from about 0.05 eq. to about 0.4 eq. based on the amount of the compound of Formula (F) employed, more preferably, about 0.2 eq. based on the amount of the compound of Formula (F) employed, of a hydrogenation catalyst, for example, any form of Pd metal on active charcoal and Ru. In some embodiments it is preferred to employ palladium on charcoal (Pd/C), and more preferably 10% dry Pd/C, and to carry out the hydrogenation reaction under a hydrogen pressure at a temperature of from about 20° C. to about 100° C., preferably at a temperature of about 60° C. Following the completion of the hydrogenation reaction, the compound of Formula (FA) thus prepared is converted to the p-toluenesulfonic acid salt (or, alternatively, to the HCl or acetic acid salt, by using the appropriate acid reagent) by reacting it with p-toluenesulfonic acid monohydrate (preferably about 1-1.3 eq based on the amount of the compound of Formula (FA) present, and more preferably about 1.2 eq) and converted to the corresponding ester by refluxing the salt with an alcohol, for example, methanol, ethanol, isopropanol, butanol, t-butanol, and other alcohols having up to 10 carbon atoms, to obtain the corresponding ester, for example, methanol to obtain the methyl ester, as illustrated in the process present in Scheme 1, the compound of Formula (FF). The ester thus obtained in this step is preferably recovered as a solid, preferably after precipitating from the reaction solution with EtOAc, 2-methyl-tetrahydrofuran, and MTBE.

As shown in Scheme 1, the compound of Formula (FF) is converted from the ester to the corresponding amide of Formula (G), by adding the solid compound of Formula (FF) to a cold (preferably below about 5° C.) solution comprising up to about 30 equivalents of ammonia dissolved in methanol, then optionally adding up to about 5 equivalents (based on the amount of the compound of Formula (FF) employed) of ammonium hydroxide, preferably, when ammonium hydroxide is added, about 2.5 equivalents of ammonium hydroxide, and agitating the mixture while maintaining it at a temperature of from about (5)° C. to about 70° C., preferably a temperature of from about (0)° C. to about (−5)° C. After the amination reaction is completed, the solution is concentrated and redissolved in water and alcohol, preferably methanol. The amino group in the compound of Formula (G) is then protected by treating the solution with a base, for example, K₂CO₃. In some embodiments using K₂CO₃ it is preferred to employ from about 0.5 equivalents of K₂CO₃ to about 2 equivalents of K₂CO₃, preferably about 0.67 eqivalents of K₂CO₃, then adding (Boc)₂O (preferably about 1 eq. to about 3 eq., more preferably about 1.4 eq). In some embodiments employing K₂CO₃, it is preferred to maintain the temperature of the mixture at a temperature of from about (0)° C. to about (40)° C., more preferably at a temperature of from about (15)° C. to about (25)° C. It will be appreciated that other acid labile protecting groups can be used in place of Boc to protect the amino functional group by employing methods known in the art. As mentioned above, the amide compound of Formula (G) is a racemic mixture of two enantiomers.

In some embodiments, the protected amide compound of Formula (G) thus obtained is converted to the compound of Formula I (H-cmpd in Scheme 1) in the form of a salt by heating a solution of the compound of Formula (G) in a solution of HCl in alcohol. In some embodiments it is preferred to employ an isopropanol HCl solution to deprotect the compound of Formula (G)

In some embodiments employing Scheme 1, it is preferred to carryout the various steps using the methodology indicated in each step of Scheme 1A, each of which have been individually discussed in detail above.

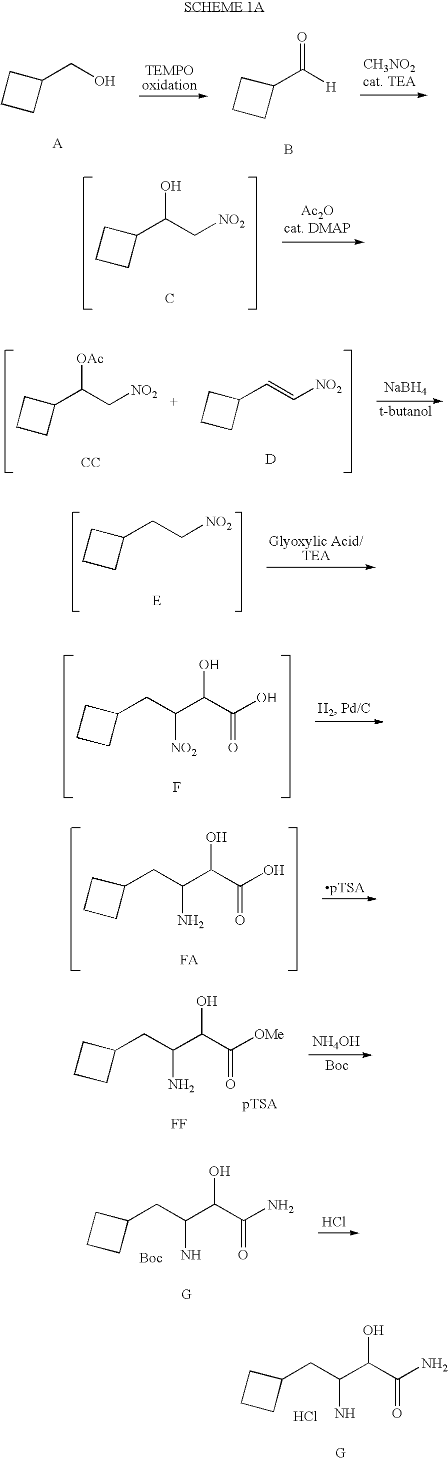

As presented above in Scheme 2, when this method of preparing the compound of Formula (G) is employed, it is preferred to carry out the conversion of the compound of Formula (C) to the compound of Formula (E) using the following reaction scheme:

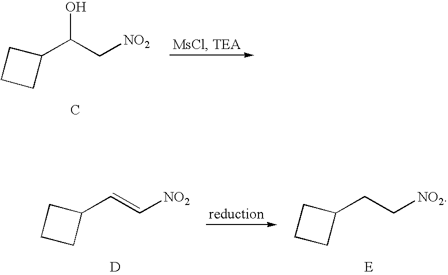

Accordingly, a solution comprising the compound of Formula (C), prepared as indicated above, is diluted with a solvent, for example, toluene (about 3-5×, preferably about 4×). To the reaction mixture, methanesulfonyl chloride (MsCl, CH₃SO₂Cl), in an amount of from about 0.9 equivalents based on the amount of the compound of Formula (C) present, to about 2 equivalents based on the amount of the compound of Formula (C) present, preferably 1.2 equivalents, is added slowly to maintain the temperature of the reaction mixture, during the mildly exothermic reaction which occurs. Triethyl amine (TEA), preferably from about 1 equivalent to about 3 equivalents, more preferably about 2.2 eq, is slowly added while maintaining the reaction mixture at a temperature below about (−25)° C. The mixture is agitated following TEA addition while maintaining the mixture at a temperature of from about (−78)° C. to about (0)° C., preferably maintaining the temperature of the mixture at from about (−30)° C. to about (−25)° C. In some embodiments it is preferred to recover the compound of Formula (D) thus prepared in the form of a toluene solution which can be employed as prepared in the next step, the preparation of the compound of Formula (E).

The compound of Formula (E) can be prepared by adding a catalytic amount of a hydrogenation catalyst, for example, a form of Pd on active charcoal or Ru to a solution comprising the compound of Formula (D), and hydrogenating the mixture. In some embodiments it is preferred to employ as a hydrogenation catalyst Pd/C, more preferably 10% dry Pd/C, and carrying out the hydrogenation reaction at a temperature of from about 0° C. to about 40° C., more preferably at a temperature of about 25° C. After hydrogenation, the reaction mixture is purified by Kugelrhor distillation to obtain pure (E). In some embodiments of this reaction step, it is preferred to dilute a solution comprising the compound of Formula (D) with alcohol, preferably isopropanol (IPA), preferably with an amount of IPA equal to from about 2× to about 5×, more preferably about 3×IPA, and a catalytic amount of a hydrogenation catalyst, as described above. In some embodiments it is preferred to employ as a hydrogenation catalyst 10% Pd/C, for example, E101R from Degussa, and to carry out the hydrogenation reaction at a temperature of from about 0° C. to about 40° C., more preferably hydrogenating at a temperature of about 25° C. In some embodiments it is preferred to employ the resultant solution of (E) in subsequent steps without further purification.

With reference to Scheme 2, presented above, in some embodiments it is preferred to carry out the various steps presented using the methodology indicated in each step of Scheme 2A, each of which have been individually discussed in detail above.

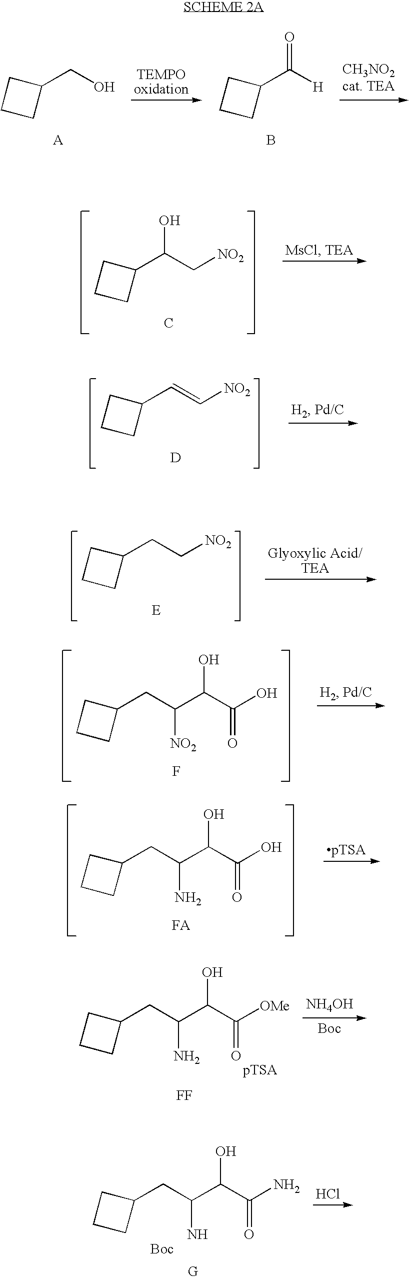

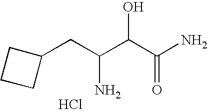

The inventors have surprisingly found that a good separation of diastereomers into enantiomer pairs can be obtained at the nitro-hydroxy acid stage of the synthesis, with reference to either Scheme 1 or Scheme 2 presented above, at the formation of the compound of Formula (F), by treating the mixture, which, as mentioned above, is a mixture of the compounds enantiomeric pairs of the compounds of Formulae F″ and F′ with either dicyclohexylamine (DCHA) or benzylamine (BnNH₂) to precipitate the desired enantiomer.

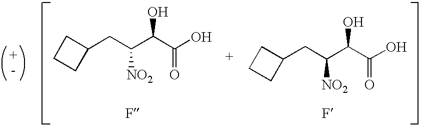

Surprisingly, the inventors have found that the ratio of major to minor diastereomers precipitated can be varied by varying the amount of time the solution is agitated with the selected amine prior to precipitation and filtration to recover the precipitate. Moreover, the inventors have surprisingly found that carrying out the reaction sequence presented in either of Schemes 1 or 2 for obtaining the compound of Formula I with a diastereomerically-enriched form of the nitro-hydroxy acid compounds either of Formula F′ or F″ results in an additional decrease in the amount of the minor isomer present in the isolated product. Accordingly, for example, precipitation of the F′ enantiomeric pair of isomers as the benzylamine salt typically results in a ratio of from about 20:1 to about 13:1 F′ enantiomeric pair of isomers relative to the amount of the F′ enantiomeric pair of isomers present in the precipitate. Reduction of a hydroxyl acid solution containing the F′ enantiomeric pair as the major diastereomers in the mixture and the F′ enantiomeric pair as the minor diastereomers present in the mixture, with subsequent conversion of the reduced compound to the corresponding F′F-HCl compound results in the enentiomeric pair of F″F isomers being present in the F′F-HCl product at a level of less than about 1%.

The inventors have surprisingly found that benzyl amine salt precipitation can provide an amount of the SR and RS salt form of the nitro-hydroxy acid compound of Formula F′ exceeding the amount of the SS and RR isomers present in an equilibrium solution of all diastereomers. Thus, the amount of the SR and RS isomer which can be precipitated from the mixture of diasteromers can exceed the amount present in an equilibrium mixture. Without wanting to be bound by theory, it is believed that the SS and RR diastereomers present in the mixture are interconverted to an SR or RS form in situ by equilibration, and then selectively precipitated in the SR and RS forms in the presence of benzyl amine (dynamic precipitation), thus providing an increase in the amount of the enantiomeric pair precipitated.

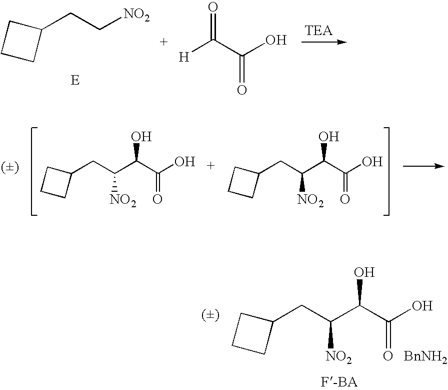

The inventors have found similar results for the dicyclohexylamine salt, wherein the F″ enantiomeric pair of diastereomers are selectively precipitated as the DCHA salt, typically from about 1:9 to about 1:14 minor:major ratio of enantiomeric pairs of diastereomers are precipitated. Without wanting to be bound by theory, it is believed that long agitation times and/or multiple precipitations will provide the major enantiomeric pair of diastereomers as a precipitate in very highly diastereomer enrichment selected for the major pair of diastereomers in accordance with the principles set forth herein. Accordingly, by carrying out the previously described synthetic steps with the precipitated diastereomerically enriched enantiomer pairs, the enantiomer pair of Formula IA,

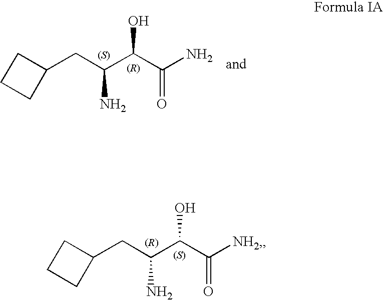

or the enantiomer pair of Formula IB,

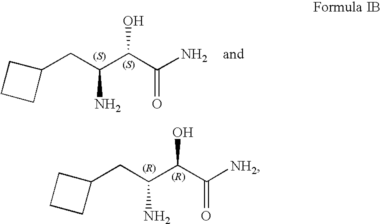

can selectively be prepared in high diastereomeric excess. Accordingly, by isolating an enantiomeric pair of diastereomers of the nitro-hydroxy-acid, the compound of Formula F (either F′ or F″), the compound of Formula I can be prepared according to the reaction schemes described above with high diastereoselectivity. To prepare an enantiomeric pair of diastereomers of the nitro-hydroxy acid, (F′-BA or F″-BA), purified compound of Formula (E) is reacted with glyoxylic acid (about 0.95-1.25 eq, preferably about 1.0 eq based on the amount of the compound of Formula (E) present) and TEA (about 1-2 eq, preferably about 1.5 eq) in a solvent, for example, a mixture of toluene and IPA. In some embodiments it is preferred to maintain the temperature of the reaction mixture in a range of from about 0° C. to about 40° C., preferably at a temperature of about 25° C., during the addition of TEA. After TEA addition, the mixture is agitated. In some embodiments it is preferred to agitate the mixture, while maintaining the mixture at a temperature of from about 0° C. to about 40° C., preferably at a temperature of from about 20° C. to about 30° C. Benzylamine (about 1-2 eq, preferably about 1.5 eq) is added and selective precipitation of the salt gives a 1:13 mixture of isomers of (F′-BA).

Alternatively, a crude solution of the compound of Formula (E) in a solvent, for example, a mixture of toluene and an alcohol, for example methanol, ethanol and isopropanol, is charged with glyoxylic acid (50% in water, about 1-2 eq, preferably 1.5 eq) and TEA is added (about 1.5-2 eq, preferably about 2 eq). After stirring at a temperature range from about 0° C. to about 40° C., preferably at about 20° C., the solvent is concentrated and the mixture redissolved in a solvent, for example, methyl tertiary-butyl ether (MTBE). Dilute HCl is added and the mixture is extracted and redissolved in a solvent such as toluene. TEA (about 1-2 eq, preferably about 1.5 eq) is added, followed by benzylamine (about 1-2.5 eq, preferably about 2 eq). Precipitation of the salt gives a 1:16 mixture of isomers of (F′-BA).

As will be appreciated from the present specification, other ratios of isomers can be obtained by varying the amount of agitation time and number of precipitations carried out with the selected amine.

(F′-BA) is acidified with a dilute solution of HCl in water and the free amino-acid compound is extracted into an organic solvent, for example, MTBE. The solvent is stripped and the residue is redissolved in an alcohol, for example, methanol, ethanol and isopropanol, and the compound of Formula (F′-BA) is reduced by hydrogenation with Pd/C, preferably 10% Pd/C (50% wet) to obtain the pair of enantiomers (F′A). (F′A) is treated with hydrogen chloride in methanol to obtain the pair of enantiomers of Formula (F′F-HCl).

F′F-HCl) is converted to the pair of enantiomers of Formula (G′) by one of two methods. Solid (F′F-HCl) is dissolved in an alcohol, preferably methanol, and reacted with NH₄OH (up to 5×, preferably 3×), while maintaining the temperature between about (−5)° C. and about 70° C., preferably at about 10° C. The resultant product is treated with a base, for example, K₂CO₃, and then the amino-group of the product is protected with an acid labile protecting group, for example, Boc. In some embodiments it is preferred to introduce the protecting group by treating the compound with (Boc)₂O (about 1-3 eq, preferably about 1.1 eq). Alternatively, solid (F′F-HCl) is reacted in a pressure bomb with a solution of ammonia in methanol, then treated with a base and a protecting group as described above.

A pair of enantiomers a compound of Formula IA is obtained by adding an alcohol (4-10×, preferably 8×) to the compound of Formula (G′), then reacting the mixture with HCl in alcohol (1-4×, preferably 2×, 5-6 N). In some embodiments it is preferred to employ IPA as the alcohol. Alternatively, HCl gas can be used.

In another alternative, (F′F-HCl), is treated with NH₄OH as described above, the product is extracted (preferably with THF, 2-methyl-THF and brine), and the organic solution is treated with HCl as described above to obtain the compound of Formula (IA).

The dicyclohexylamine salt comprising the enantiomer pair of Formula (F″), (F″-DCHA), is prepared by dissolving the compound of Formula (E) in a solvent, for example, a mixture of toluene and ethanol, and adding glyoxylic acid or glyoxylic acid monohydrate (about 1-2 eq, preferably about 1.2 eq), followed by dicyclohexylamine (about 1-2 eq, preferably about 1.5 eq). The resultant precipitate recovered is a 14:1 mixture of the enantiomeric pair of diastereomers, with the SS and RR isomers predominating, the enantiomeric pair of Formula (F″-DCHA). The enantiomeric pair of diastereomers of the Formula (F″-DCHA) obtained are treated in a manner similar to that described for the enantiomeric pair of diasereomers of the Formula (F′-BA) to obtain the enantiomeric pair of diastereomers of Formula (IB),

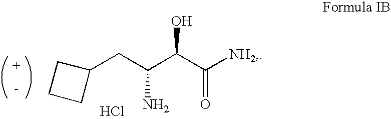

Those skilled in the art will appreciate that well known extraction procedures can be used to purify the intermediates as necessary in the various steps described above.

Following are detailed examples showing typical procedures for preparing the compound of formula I and the intermediates employed in the process.

### Example 1

### Preparation of B

To a solution of compound A (cyclobutanemethanol) (100 g) in CH₂Cl₂ (1 L) was added a solution of 24% KBr in water (62.5 ml). To this mixture was added a solution of NaHCO₃ in water (150 ml), and the mixture was cooled to −5° C. To this was added TEMPO reagent (1.8 g), and the mixture stirred for 20 min. Slowly 5% NaOCl (about 1900 ml) was charged to the mixture while maintaining the temperature between −5° C. to 0° C. The mixture was then stirred for about 30 min. Then a solution of KH₂PO₄ in water (8.25%, 400 ml) was added and the mixture stirred for about an additional 30 min while warming to RT. The layers were then split and separated. The organic layer was dried with anhydrous MgSO₄ and filtered. The dry organic layer was carefully distilled under partial vacuum (about 110 Torr) with a bath temperature about 0° C. to remove most of the solvent. The concentrate was further distilled at atmospheric pressure to obtain compound B (cyclobutanecarboxaldehyde) (70.6 g; 70.4%, 96% pure) as a colorless liquid: ¹H NMR (400 MHz, CD₃OD) [symbol] 9.65 (1H, s), 3.05 (1H, m), 2.13 (2H, m), 2.03 (2H, m), 1.95 (1H, m), 1.80 (1H, m).

### Example 2

### Preparation of C

Under nitrogen, a mixture of nitromethane (39 ml), TEA (25 ml) and toluene (200 ml) was agitated for about 10 min at a temperature around 15° C. To the mixture was slowly added compound B (50 g), at 15-25° C. and the mixture was agitated at 20-25° C. for 16.5 hrs. The resulting solution of compound C was used in situ for the next step. ¹H NMR (400 MHz, CDCl₃) δ 4.40-4.33 (1H, m), 4.32-4.24 (2H, m), 2.47-2.37 (1H, m), 2.14-1.80 (6H, m).

### Example 3

### Preparation of CC/D

The solution of C from Example 2 was cooled to 0-5° C. and solid DMAP (3.6 g) was added. The reaction mixture was stirred for about 10 min to dissolve all solids, and then acetic anhydride (75.5 ml, 1.35 eq.) was slowly added at around 15° C. After stirring at 15-20° C. for 2 hr, the resulting solution of compounds CC/D was used in situ for the next step.

### Example 4

### Preparation of E, Method I

A solution of CC/D prepared from compound B (Example 3, 5.0 g) was charged with 5% Pd/C (50% wet, 2.0 g, E101 R from Degussa), CH₃OH (10 ml) and TEA (5 ml), and the mixture stirred under hydrogen pressure (˜5 psi) at around 0° C. Reaction was over after 16 h (determined by HPLC analysis). The reaction mixture was charged with Celite (0.3 g), agitated for 30 min, then filtered and washed with toluene (30 mL). The resulting organic layer was washed with aq. 1N HCl (10 mL), aq. sat'd NaHCO₃ (10 mL) and brine (10 mL). The resulting organic solution contains compound E, (5.23 g, 67.9% yield). ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 5

### Preparation of E, Method II

A solution of CC/D from Example 3 (10 g) was charged with PEG-400 (10 ml) and water (6 ml) and cooled to around 0° C. Solid NaBH₄ (8.6 g) was charged slowly at 5-20° C. using solid-charging apparatus and the mixture stirred for 40 min at around 20° C. The resulting slurry was cooled to 0-5° C. and quenched with cold water (40 ml) slowly at 0-10° C. The organic layer was separated and the aqueous layer was back-extracted with toluene (120 ml). The organic solution contained compound E (11.1 g, 72% yield). ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 6

### Preparation of E, Method II

A solution of CC/D prepared in Example 3 (50 g) was charged with PEG-400 (50 ml) and water (32 ml) and cooled to around 0-5° C. The reaction mixture was cooled below 5° C. In another reactor were charged NaBH4 (solid, 53.75 g, 2.5 eq.) and toluene (200 ml), and the slurry was cooled below 5° C. The CC/D solution was transferred into the NaBH₄ suspension over 1 hr, while maintaining a batch temperature below 20° C. The temperature was adjusted to 20° C., the mixture agitated for 1 hr, and a small sample was taken for HPLC analysis. Once reaction was completed as determined by HPLC, the reaction mixture was cooled below 5° C. The batch was slowly charged into cold water (250 ml) while maintaining a temperature below 20° C. The batch temperature was adjusted to 20° C. and the mixture agitated for 30 min, then the organic layer was separated. The aqueous layer was back-extracted with toluene (300 ml). The combined organics were washed with 1N of HCl (250 ml), then sat'd NaHCO₃ (250 ml), then brine (250 ml). The product solution was concentrated to 275 ml at a temperature below 30° C. under vacuum. The resulting solution was used for the next step. The organic solution contained compound E (50.7 g, 66% yield). ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 7

### Preparation of E, Method III

A solution of CC/D prepared in Example 3 (50 g) was charged t-BuOH (95 ml) while maintaining a temperature between 5 and 15° C. The reaction mixture was cooled to a temperature between 0 and 10° C. NaBH₄ (solid, 32.25 g, 1.5 eq.) and toluene (200 ml) were charged in another reactor (3 L reactor for 50 g scale). The slurry was cooled to a temperature between 0 and 10° C. The CC/D mixture was transferred into the NaBH₄ suspension over 1-3 hr, while maintaining a batch temperature between 15 and 25° C. The temperature was adjusted to between 15 and 25° C. and the mixture was agitated for 4-6 hr. A small sample was taken (IPC) for HPLC analysis. Once reaction was done as determined by HPLC (spec <1%), the reaction mixture was cooled to a temperature between 0° C. and 10° C. The mixture was added to cold water (250 ml) slowly while maintaining a temperature between 15° C. and 25° C. The temperature was adjusted to between 15° C. and 25° C. and the mixture agitated for 30 min. The organic layer was separated and the aqueous layer was back extracted with toluene (200 ml). The combined organics were washed with 1N of HCl (150 ml), sat'd NaHCO₃ (150 ml), then brine (150 ml). The organic solution contained compound E (52 g, 68% yield). The resulting solution was used for the next step. ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 8

### Preparation of D from B Via MsCl

To a solution of nitromethane (16.8 g) in toluene (48 ml) at RT, TEA (4.2 g) was added slowly and the mixture was agitated for about 10 min. To the mixture, compound B (11.6 g) was slowly added. The reaction mixture was stirred at RT for about 6 h, after which the reaction was monitored for completion by NMR. The solution was diluted further with toluene (50 ml). The resulting solution was cooled below −25° C. CH₃SO₂Cl (18.9 g) was slowly added over about 10 min at −30 to −25° C. TEA (30.7 g) was slowly charged; the addition was regulated to keep exotherm below ±25° C. The reaction mixture was agitated for about 10 min at ±30 to −25° C. The reaction mixture was warmed to about −5 to 0° C. and charged with water (100 ml) slowly.

The reaction mixture was agitated for about 5-15 min at RT, settled and split. The aqueous layer is back-extracted with about toluene (50 ml). The combined organics were washed with 1N HCl (72 ml). The layers were split and separated. The organic layer was then washed with saturated NaHCO₃ (72 ml). The layers were split and separated. The organic was then washed with saturated brine (2×72 ml). The layers were split and separated to give a solution of compound D. ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 9

### Preparation of E

To the solution of compound D in toluene from Example 8 was added dry palladium on carbon catalyst (2.1 g, 10% active) and the resulting mixture was hydrogenated with a balloon for 6-8 h. The reaction progress was monitored by HPLC. After reaction completion, the catalyst was filtered and washed with toluene (18 ml). The product solution was concentrated to a neat liquid. The concentrate was further purified by Kugelrohr distillation to obtain clean, colorless compound E (18.9 g, 75% from aldehyde B). ¹H NMR (400 MHz, CDCl₃) δ 4.31 (2H, t, J=7.2), 2.39-2.30 (1H, m), 2.15-2.07 (4H, m), 1.98-1.82 (2H, m), 1.73-1.64 (2H, m).

### Example 10

### Preparation of E

The solution of compound D from Example 8 was diluted with IPA (32 ml). The resulting solution was added to 50% wet palladium on carbon catalyst (1.8 g, 10% active) and the resulting mixture was hydrogenated with a balloon for 18 h. The reaction progress was monitored by HPLC. After reaction completion, the catalyst was filtered and washed with toluene (35 ml). This solution contained 10.6 g of compound E. The solution was concentrated to 85 mL at a temperature below 30° C. under vacuum. Toluene (35 ml) was added and the mixture concentrate to 85 mL again. Toluene was added to make 106 ml Of the solution, and IPA (10.6 ml) was added. This solution contained 9.8 g of compound E (55% yield from B) and used in situ for the next step. ¹H NMR (400 MHz, CDCl₃) δ 7.36 (1H, dd, J=7.4, 13.4), 6.93 (1H, dd, J=13.4, 1.4), 3.18 (1H, m), 2.31-2.25 (2H, m), 2.09-1.93 (4H, m).

### Example 11

### Preparation of F

Into a solution of compound E from Example 7 (111.5 g, 0.155 mol in toluene with 16-20% nitromethane) was charged CH₃OH (335 ml). The mixture was cooled to a temperature between 0 and 10° C. Glyoxylic acid (256 g, 2.0 eq., 50% in water) was added while maintaining a temperature between 0 and 20° C. The mixture was agitated for 10 min. TEA (327 m, 2.7 eq.) was added slowly while maintaining a temperature between 15 to 25° C. The mixture was agitated for 15-20 hrs at a temperature between 25 and 35° C. and reaction completion was monitored by HPLC (spec is <2% by area). Upon completion, the reaction mixture was cooled to a temperature between 0 and 10° C. Aq. K₂CO₃ (558 ml of 2.5% K₂CO₃ by weight in water) was added while maintaining a temperature between 0 and 10° C., and the mixture was agitated for 15 min at a temperature between 5 and 15° C. The aqueous layer containing compound F was separated and cooled to a temperature between 0 and 10° C. Conc. HCl (175 ml) was added until pH was 1.5-2, while maintaining a temperature between 0 and 10° C. MTBE (223 ml) was added and the mixture agitated for 10 min at a temperature between 10 and 20° C. The layers were split and the aqueous layer was back-extracted with MTBE (167 ml). The combined organics were washed with 1N HCl (112 ml), brine (112 ml, 10% NaCl in water), and another wash with brine (56 ml, 10% NaCl in water). The resulting organic solution of compound F is used for the next step without concentration/distillation. ¹H NMR (400 MHz, DMSO-d6) δ 4.64 (1H, m), 4.46 (0.5H, d, J=4.0), 4.24 (0.5H, d, J=6.2), 2.24 (2H, m), 1.99 (2H, m), 1.83 (3H, m), 1.60 (2H, m).

### Example 12

### Preparation of FF

Into 10% Pd/C (10.4 g, 50% wet) in a hydrogenator was added dry CH₃OH (155 ml). The free acid solution of compound F (51.8 g) prepared in Example 11 was transferred into a hydrogenator. The resulting slurry was hydrogenated under hydrogen pressure (90 psi) at 60 deg. After 15 h, p-toluenesulfonic acid monohydrate (58.1 g) was added and stirred for 30 min to dissolve solid product. Celite (5.2 g) was added and the mixture agitated for 15 min. The slurry was filtered and washed with CH₃OH (78 ml). The resulting solution was distilled to about 140 ml of volume. CH₃OH (518 ml) was added and the resulting solution was distilled to a minimum volume, resulting in a slurry. CH₃OH (518 ml, KF spec <3%) was added and the mixture was heated to reflux for 16 hr. The mixture was concentrated to a volume of 207 ml under atmospheric pressure. EtOAc (518 ml) was added and the mixture was concentrated to a volume of 414 ml under atmospheric pressure. The resulting slurry was cooled to 0-5° C. MTBE (259 ml) was added and the resulting slurry was cooled to −13 to −10° C. and agitated for 1 hr. The product was filtered and washed with a 1:1 mixture of EtOAc and MTBE (104 ml) and dried to give 76.8 g of solid (54:46 ratio of two diastereomers, 84% yield). ¹H NMR (400 MHz, DMSO d₆) [symbol] 7.91 (1.62H, br s), 7.82 (1.38H, br s), 7.48 (2H, m), 7.12 (2H, d, J=7.8), 6.52 (0.46H, d, J=5.2), 6.33 (0.54H, d, J=5.2), 6.33 (0.54H, d, J=5.2), 4.38 (0.54H, dd, J=2.9, 5.1), 4.16 (0.46H, m), 3.71 (1.5H, s), 3.70 (1.5H, s), 3.27 (0.54H, m), 3.21 (0.46H, m), 2.39-2.30 (1H, m), 2.30 (3H, s), 2.02 (2H, m), 1.78 (2H, m), 1.60 (3H, m), 1.50 (1H, m).

### Example 13

### Preparation of G

Into compound FF (30.0 g) was added 7N NH₃/CH₃OH (93.8 ml), pre-chilled to below 5° C. The resulting slurry was cooled to a temperature between −5 and 5° C. NH₄OH (75.0 ml) pre-chilled to below 5° C., was added while maintaining a temperature between −2 and 5° C. The mixture was agitated for 1.5 day, while maintaining a temperature between 0 and 5° C. The mixture was concentrated to a minimum volume under vacuum below 40° C. CH₃OH (60 ml) and water (60 ml) were added while maintaining a temperature between 15 and 25° C. K₂CO₃ (7.7 g, 0.67 eq.) was added and the mixture agitated for 1% min, then (Boc)₂O (25.6 ml, 1.4 eq.) was added while maintaining a temperature between 15 and 25° C. The mixture was agitated for 15 hr at a temperature between 15 and 25° C. Water (240 ml) was added while maintaining a temperature below 20° C., the resulting slurry was cooled to 5° C. and the mixture agitated for 17 hr. The product was filtered and washed with water (25 ml) and dried to give 19.5 g of solid (86% yield), a mixture of two diastereomers (SS/RR: SR/RS=55:45). ¹H NMR (400 MHz, DMSO d₆) [symbol] 7.19 (2H, m), 6.28 (0.55H, d, J=9.2), 5.93 (0.45H, d, J=9.4), 5.44 (1H, m), 3.82 (0.55H, m), 3.74-3.61 (1.45H, m), 2.27 (1H, m), 1.97 (2H, m), 1.76 (2H, m), 1.61-1.24 (4H, m), 1.38 (4.95H, s), 1.36 (4.05H, s).

### Example 14

### Preparation of F′-BA, Enantiomeric Pair of Diastereomers,

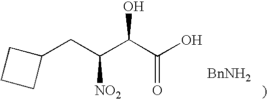

Compound E (50.0 g) was dissolved in a mixture of toluene (450 ml) and IPA (50 ml). Glyoxlic acid monohydrate (1.0 eq., 35.65 g) was added and the mixture agitated for 10 min. TEA was charged (1.5 eq., 81.5 ml) while maintaining the temperature between 15 to 20° C. When the reaction was complete (6-12 hr), benzylamine (1.5 eq., 63.4 ml) was added. MTBE (500 ml) was added and the mixture agitated for 24 h. The precipitate was filtered and washed with MTBE (100 ml) and dried under vacuum at room temperature overnight. The benzylamine salt was obtained in 82% yield (98 g) as a mixture of part 1 and part 2 isomers (1:13).: ¹H NMR (400 MHz, DMSO d₆) δ 7.45 (5H, m), 6.65 (2H, broad), 4.25 and 4.5 (1H, wide dd, ratio=13:1), 4.0 (2H, s), 3.75 (1H, m), 2.4 (2H, m), 2.1 (2.5H, m), 1.9 (2.5H, m), 1.7 (2H, m).

### Example 15

### Preparation of F′-BA, Enantiomeric Pair of Diastereomers

To a solution of crude compound E from Example 6 (55.0 g, 426 mmol) in toluene was added ethanol (275 ml). The resultant mixture was cooled below 10° C. 50% glyoxylic acid in water (94.6 g, 1.5 eq.) was added slowly at a temperature below 20° C. TEA (119.35 ml, 2 eq.) was added at 15-20° C., and after stirring at about 20° C. for 4 h, the reaction mixture was concentrated to about 275 ml at a temperature below 30° C. MTBE (550 ml) was added, and the resultant mixture was cooled to about 10° C. A dilute HCl solution (prepared from 110 ml of concentrated HCl and 440 ml of water) was added at a temperature below 20° C. The organic layer was separated and washed with brine (165 ml). Toluene (550 ml) was added, and the resultant mixture was cooled to about 10° C. TEA (89.65 ml, 1.5 eq.) was added at 10-20° C. Benzylamine (92.95 ml, 2 eq.) was added at 15-20° C. The resultant slurry was stirred at about 20° C. for 19.5 h. The precipitate was filtered, washed with MTBE (about 300 ml), and dried under vacuum at RT overnight. The benzylamine salt was obtained in 84% yield (111.2 g) as a mixture of part 1/part 2 isomers (1/16). ¹H NMR (400 MHz, DMSO d₆) δ 7.45 (5H, m), 6.65 (2H, broad), 4.25 and 4.5 (1H, wide dd, ratio=16:1), 4.0 (2H, s), 3.75 (1H, m), 2.4 (2H, m), 2.1 (2.5H, m), 1.9 (2.5H, m), 1.7 (2H, m).

### Example 16

### Preparation of Dicyclohexylamine Salt Enantiomeric Pair of Diastereomers, (F-DCHA)

Compound E (6.46 g, 50 mmol) was dissolved in a mixture of toluene (58 ml) and ethanol (6.5 ml). Glyoxylic acid monohydrate (5.52 g, 1.2 eq.) was added at RT. Dicyclohexylamine (13.6 g, 1.5 eq.) was added slowly at a temperature below 25° C. After stirring at RT for 3 h, to the resultant slurry was added MTBE (65 ml) at RT. After 2 h, the precipitate was filtered, washed with MTBE, and dried under vacuum. The dicyclohexylamine salt was obtained in 50% yield (9.63 g) as a mixture of part 1/part 2 isomers (9/1). ¹H NMR (400 MHz, DMSO d₆) δ 4.34-4.61 (1H, ddd and dt, ratio=1:9), 3.73 and 4.01 (1H, d, ratio=1:9), 3.05 (2H, m), 2.10-2.23 (2H, m), 1.65-1.84 (6H, m), 1.90-2.04 (5H, m), 1.45-1.65 (5H, m), 1.20-1.33 (8H, m), 1.03-1.15 (2H, m)

### Example 17

### Preparation of Dicyclohexylamine Salt Enantiomeric Pair of Diastereomers, (F″-DCHA)

Compound E (1.0 g, 7.7 mmol) was dissolved in a mixture of toluene (9 ml) and ethanol (1 ml). Glyoxylic acid monohydrate (0.71 g, 1 eq.) was added at RT. Dicyclohexylamine (2.31 ml, 1.5 eq.) was added slowly at a temperature below 25° C. After stirring at RT for 16 h, to the resultant slurry was added toluene (10 ml) at RT. After 24 h, the precipitate was filtered, washed with toluene, and dried under vacuum. The dicyclohexylamine salt was obtained in 43% yield (1.29 g) as a mixture of part 1/part 2 isomers (14/1). ¹H NMR (400 MHz, DMSO d₆) δ 4.34-4.61 (1H, ddd and dt, ratio=1:14), 3.73 and 4.01 (1H, d, ratio=1:14), 3.05 (2H, m), 2.10-2.23 (2H, m), 1.65-1.84 (6H, m), 1.90-2.04 (5H, m), 1.45-1.65 (5H, m), 1.20-1.33 (8H, m), 1.03-1.15 (2H, m).

### Example 18

### Preparation of F′F-HCl Salt, Single Diastereomer, (1:1 Mixture of Enantiomers of RS and SR)

To a dilute solution of HCl in water (438 ml, 1 N), at 15-20° C., was added the compound of Example 15 (100 g) of predominantly one diastereomer, and the mixture was stirred until all dissolved. MTBE (300 ml) was added and the mixture was stirred. The layers were settled and split. The aqueous layer was extracted a second time with MTBE (200 ml). The combined organics were washed with 10% aqueous NaCl solution (50 ml). The layers were settled and split, and the organic layer was concentrated to a minimum volume under reduced pressure. MTBE (300 ml) was added to the resultant oil and concentrated again to a minimum volume under reduced pressure. The resultant oil was dissolved in CH₃OH (200 ml) and the solution was concentrated to about 150 ml under reduced pressure. The resultant residue was diluted with CH₃OH (500 ml), and to this solution was added 10% Pd—C (50% wet) (6.5 g) and kept at 90 psi of hydrogen at a temperature of about 50 to 60° C. After the reaction was complete, anhydrous HCl in CH₃OH was added and the mixture stirred for about 30 min. The catalyst was filtered and washed with CH₃OH (65 ml) to give a solution of compound FF-HCl. ¹H NMR (400 MHz, DMSO d6) δ 8.12 (3H, br s), 6.48 (1H, d, J=5.5), 4.19 (1H, m), 3.69 (3H, s), 3.15 (1H, m), 2.42 (1H, m), 2.02 (2H, m), 1.80 (2H, m), 1.71 (2H, m), 1.59 (2H, m).

### Example 19

### Preparation of Compound G′ (Pair of SR and RS Enantiomers)

Into solid FF-HCl (5 g) was charged CH₃OH (10 ml). The resulting solution was cooled to below 10° C. and NH₄OH (15 ml) was added, while maintaining temperature below 10° C. After agitating for 15 h at 10° C., the reaction mixture was concentrated to a minimum volume under vacuum at a temperature below 30° C. CH₃OH (25 ml) was added while maintaining a temperature between 15 and 25° C. Water (5 ml) was added, followed by K₂CO₃ (2.2 g), while maintaining a temperature between 15 and 25° C. After agitating for 10 min, (Boc)₂O (5.5 ml) was charged, while maintaining a temperature between 15 and 25° C. The mixture was agitated for 4 hr at a temperature between 15 and 25° C. Completion of the reaction was monitored by HPLC-2 (spec <3%). Water (40 ml) was added while maintaining a temperature below 25° C. and the resulting slurry was cooled to 10° C. and agitated for 1 hr. The product was filtered and washed with a 1:3 mixture of CH₃OH and water (25 ml) and dried to give 5.3 g of solid (87% yield). ¹H NMR (400 MHz, DMSO d₆) δ 7.21 (2H, br s), 5.94 (1H, d, J=9.5), 5.44 (1H, d, J=6.3), 3.73 (1H, dd, J=3.1, 6.2), 3.63 (1H, m), 2.27 (1H, m), 2.00 (2H, m), 1.77 (2H, m), 1.60 (3H, m), 1.43 (1H, m), 1.36 (9H, s).

### Example 20

### Preparation of G′ (1:1 Mixture of Enantiomers of RS and SR)

Into solid F′F-HCl (10 g) in a pressure bomb was charged cold 7N NH₃ in CH₃OH (200 ml). The resulting solution was agitated for 5 h at 60° C. in the pressure bomb. The reaction mixture was concentrated to a minimum volume under vacuum at a temperature below 30° C. CH₃OH (60 ml) and water (20 ml) were added, followed by K₂CO₃ (6.6 g, 1 eq.), while maintaining a temperature between 15 and 25° C. After agitating for 10 min, (Boc)₂O (11 ml, 1.1 eq.) was charged, while maintaining a temperature between 15 and 25° C. The mixture was agitated for 4-15 hr at a temperature between 15° C. and 25° C. The reaction was monitored for completion by HPLC-2 (spec <2%). Water (50 ml) was added while maintaining a temperature below 25° C., and the resulting slurry was cooled to 5° C. and agitated for 2 hr. The product was filtered and washed with a 1:2 mixture of CH₃OH and water (50 ml) and dried to give 11.2 g of solid compound G (92% yield). ¹H NMR (400 MHz, DMSO d₆) δ 7.21 (2H, br s), 5.94 (1H, d, J=9.5), 5.44 (1H, d, J=6.3), 3.73 (1H, dd, J=3.1, 6.2), 3.63 (1H, m), 2.27 (1H, m), 2.00 (2H, m), 1.77 (2H, m), 1.60 (3H, m), 1.43 (1H, m), 1.36 (9H, s).

### Example 21

### Preparation of the Compound of Formula IA—(1:1 Mixture of Enantiomers of RS and SR)

Into the compound of Example 20 (5.0 g) was added IPA (40 ml), followed by the addition of 5-6 N HCl in IPA (10 ml). The resulting slurry was heated to 50° C. and stirred for 4 hr. The slurry was cooled to RT and agitated for 1 hr. The product was filtered and washed with MTBE (25 ml) and dried to give 3.72 g of solid (97% yield). ¹H NMR (400 MHz, DMSO d₆) δ 3.98 (1H, d, J=3.7), 3.1 (1H, m), 2.4 (1H, m), 2.02 (2H, m), 1.77 (2H, m), 1.6 (4H, m).

### Example 22

### Preparation of the Compound of Formula IA—(1:1 Mixture of Enantiomers of RS and SR)

Into the compound F′-BA (Example 14, 15) (60 g) was charged pre-cooled aqueous HCl solution prepared from 240 ml of water and 22.8 ml of conc HCl. MTBE (180 ml) was added and the resulting biphasic mixture was agitated for 10 min. The aqueous layer was separated and back extracted with MTBE (120 ml). The combined organics were washed with brine (30 ml), the organic layer was separated and concentrated to a minimum volume. The resulting oil was diluted with MTBE (180 ml) and concentrated to a minimum volume. The resulting oil was diluted with CH₃OH (600 ml) and hydrogenated in a hydrogenator containing 10% Pd/C (50% wet) (2 g). The solution was then hydrogenated under Hydrogen pressure (90 psi) at 50 deg. After 15 hr, a sample was taken for HPLC (disappearance of nitro compound). When the reaction was completed, anhydrous HCl in CH₃OH (1.2 eq., prepared from 16.5 ml of AcCl in 120 ml of CH₃OH) was added. Celite (2.0 g) was added and the mixture agitate for 30 min. The resultant mixture was filtered and washed with CH₃OH (39 ml), then heated to 60 deg for 16 hr. Completion of the reaction was monitored by NMR: spec is <2%.

The resultant solution was concentrated to a volume of 80 ml and cooled below 20° C. CH₃OH (40 ml), THF (110 ml) and NH₄OH (29% in water) (80 ml) were added. After agitating for 15 h at 0° C. to RT, THF (118 ml), 2-methyltetrahydrofuran (118 ml) and brine (79 ml) were added. The mixture was agitated for 30 min. The lower aqueous layer was split and back extracted twice with a mixture of THF (118 ml) and 2-methyltetrahydrofuran (59 ml). The combined organics were concentrated to a volume of 120 ml. The resulting mixture was diluted with IPA (393 ml) and cooled to 10° C. Into the resulting slurry was added 5N HCl in IPA (53 ml). After stirring 1 hr, the product was filtered and washed with MTBE (197 ml) and dried to give 26.2 g of solid compound of formula I (69% yield).). ¹H NMR (400 MHz, DMSO d₆) δ 3.98 (1H, d, J=3.7), 3.1 (1H, m), 2.4 (1H, m), 2.02 (2H, m), 1.77 (2H, m), 1.6 (4H, m).

While the present invention has been described with and in conjunction with the specific embodiments set forth above, these examples are meant to be illustrative and not limiting. Many alternatives, modifications and other variations thereof will be apparent to those of ordinary skill in the art. All such alternatives, modifications and variations are intended to fall within the spirit and scope of the present invention.

1. A process for preparing the compound of Formula I comprising: 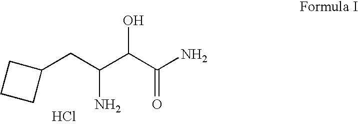
(A) coupling nitroalkane (E) with glyoxylic acid to obtain the nitro-hydroxy acid (F): 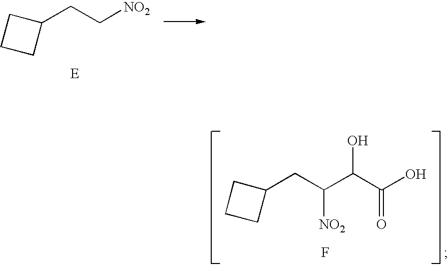
wherein (E) is prepared by a process comprising:
(a) oxidizing cyclobutanemethanol (A) with 2,2,6,6-tetramethyl-1-piperidinyloxy, free radical, to obtain cyclobutanecarboxaldehyde (B): 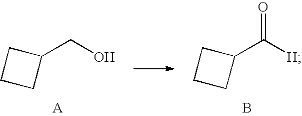
(b) coupling the aldehyde (B) with nitromethane to obtain the nitro-alcohol (c): 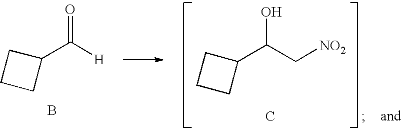
(c) converting compound (C) to compound (E) by
(1) a first method comprising:
(i) reacting nitro-alcohol (C) with acetic anhydride to obtain a mixture of compounds (CC) and (D): 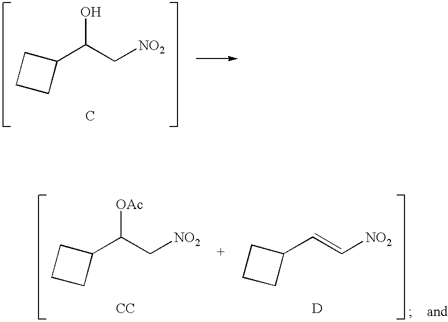
(ii) converting the mixture of (CC) and (D) obtained in Step (c)1(i) to nitroalkane (E) by a process selected from: 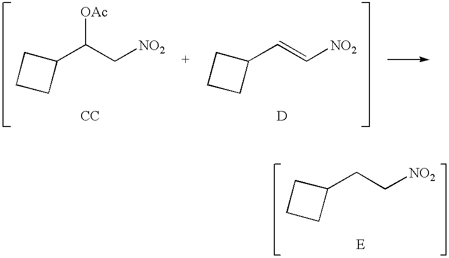
(I) hydrogenation of the mixture; or
(II) reduction of the mixture with sodium borohydride in the presence of PEG-400; or
(III) reduction of the mixture with sodium borohydride in the presence of an alcohol, or
(2) a second method comprising:
(i) reacting the nitro-alcohol (C) with CH₃SO₂Cl and triethylamine to obtain compound (D): 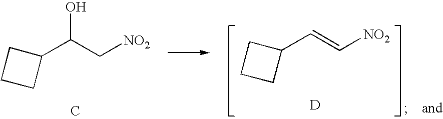
(ii) reducing (D) obtained in process Step (2)(i), thereby providing nitroalkane (E);
(B) hydrogenating compound (F) provided by Step “A” to yield amino-hydroxy acid (FA): 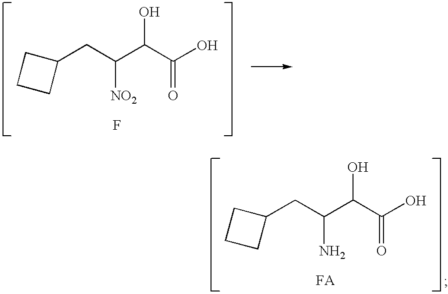
(C) refluxing (FA) with p-toluenesulfonic acid and esterifying to obtain (FF); 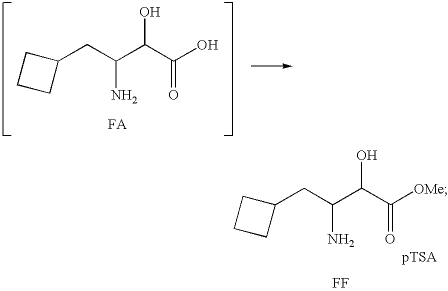
(D) converting the ester to an amide and protecting the amino group of (FF) to obtain (G), wherein Prot is a protecting group: 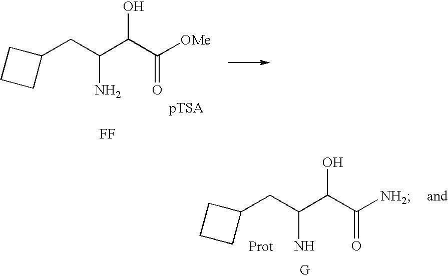
(E) heating (G) prepared in Step “D” in a solution of HCl in alcohol.

2. The process of claim 1 comprising
(i) coupling the nitroalkane (E) with glyoxylic acid to obtain the nitro-hydroxy acid (F): 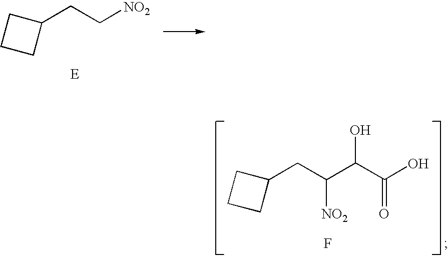
wherein (E) is prepared by:
(a) oxidizing cyclobutanemethanol (A) with 2,2,6,6-tetramethyl-1-piperidinyloxy, free radical, to obtain cyclobutanecarboxaldehyde (B): 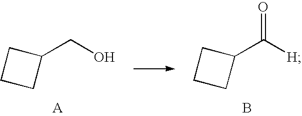
(b) coupling the aldehyde (B) with nitromethane to obtain the nitro-alcohol (c): 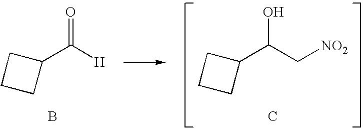
(c) reacting the nitro-alcohol (C) with acetic anhydride to obtain a mixture of compounds (CC) and (D): 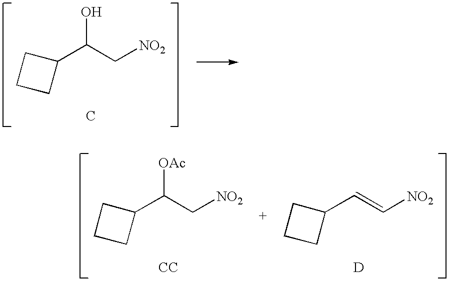
(d) converting the mixture of (CC) and (D) to the nitroalkane (E) 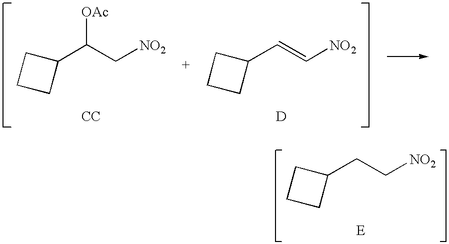
by
I) hydrogenation of the olefin;
II) reduction of the olefin with sodium borohydride in the presence of PEG-400; or
III) reduction of the olefin with sodium borohydride in the presence of an alcohol;
(ii) hydrogenating (F) to obtain the amino-hydroxy acid (FA): 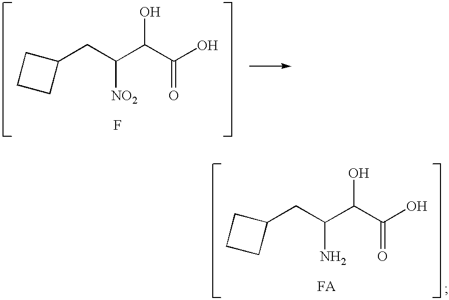
(iii) refluxing (FA) with p-toluenesulfonic acid and esterifying to obtain (FF): 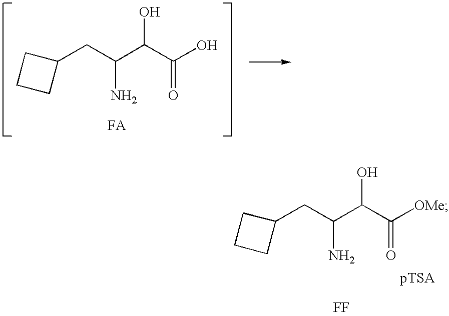
(iv) converting the ester to an amide and protecting the amino group of (FF) to obtain (G), wherein Prot is a protecting group: 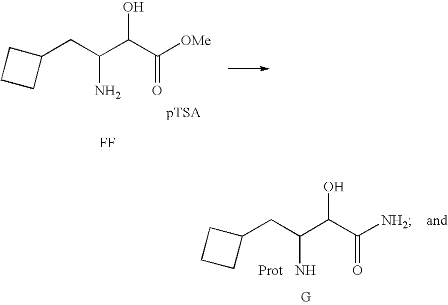
(v) heating (G) in a solution of HCl in alcohol.

3. The process of claim 1 comprising
(i) coupling the nitroalkane (E) with glyoxylic acid to obtain the nitro-hydroxy acid (F): 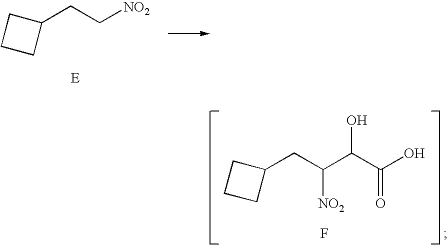
wherein (E) is prepared by:
(a) oxidizing cyclobutanemethanol (A) with 2,2,6,6-tetramethyl-1-piperidinyloxy, free radical, to obtain cyclobutanecarboxaldehyde (B): 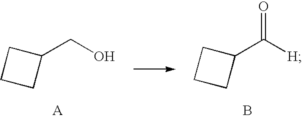
(b) coupling the aldehyde (B) with nitromethane to obtain the nitro-alcohol (C): 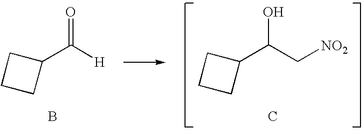
(c) reacting the nitro-alcohol (C) with CH₃SO₂Cl and triethylamine to obtain compound (D); and 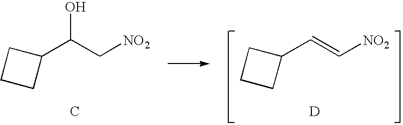
(d) reducing (D) to obtain the nitroalkane (E);
(ii) hydrogenating (F) to obtain the amino-hydroxy acid (FA): 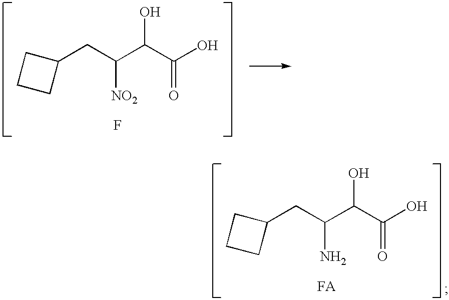
(iii) refluxing (FA) with p-toluenesulfonic acid and esterifying to obtain (FF): 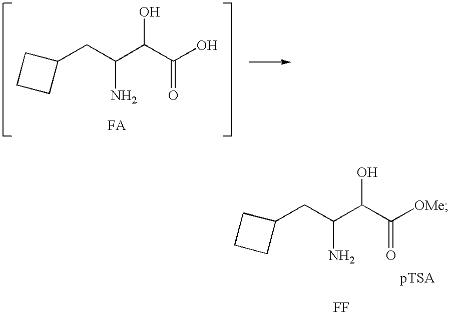
(iv) converting the ester to an amide and protecting the amino group of (FF) to obtain (G), wherein Prot is a protecting group: 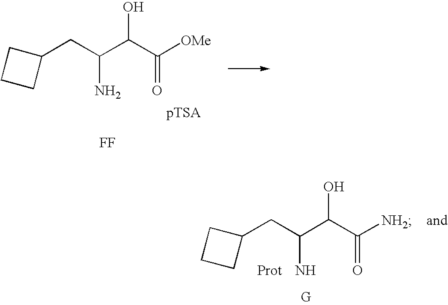
(v) heating (G) in a solution of HCl in alcohol.

4. The process of claim 2 comprising
(i) coupling the nitroalkane (E) with glyoxylic acid in the presence of TEA in toluene to obtain the nitro-hydroxy acid (F);
wherein (E) is prepared by:
(a) oxidizing cyclobutanemethanol (A) with 2,2,6,6-tetramethyl-1-piperidinyloxy, free radical, to obtain cyclobutanecarboxaldehyde (B);
(b) coupling the aldehyde (8) with nitromethane in the presence of TEA in toluene to obtain the nitro-alcohol (C);
(c) reacting the nitro-alcohol (C) with acetic anhydride and a catalytic amount of DMAP to obtain a mixture of compounds (CC) and (D);
(d) converting the mixture of (CC) and (D) to the nitroalkane (E) by:
I) hydrogenation with Pd/C in alcohol;
II) reduction of the olefin with sodium borohydride in the presence of PEG-400; or
III) reduction of the olefin with sodium borohydride in the presence of an alcohol;
(ii) hydrogenating (F) with Pd/C in alcohol to obtain the amino-hydroxy acid
(FA);
(iii) refluxing (FA) with p-toluenesulfonic acid and esterifying by refluxing in an alcohol to obtain (FF);
(iv) converting the ester to an amide by treating with NH₄OH and protecting the amino group of (FF) by treating with a base and a protecting group to obtain (G); and
(v) heating (G) in a solution of HCl in alcohol.

5. The process of claim 4 wherein the reduction in Step 1, part (d) is carried out by Method III.

6. The process of claim 3 comprising
(i) coupling the nitroalkane (E) with glyoxylic acid in the presence of TEA in toluene to obtain the nitro-hydroxy acid (F);
wherein (E) is prepared by
(a) oxidizing cyclobutanemethanol (A) with 2,2,6,6-tetramethyl-1-piperidinyloxy, free radical, to obtain cyclobutanecarboxaldehyde (B)
(b) coupling the aldehyde (B) with nitromethane in the presence of TEA in toluene to obtain the nitro-alcohol (C);
(c) reacting the nitro-alcohol (C) with CH₃SO₂Cl and triethylamine to obtain compound (D); and
(d) reducing (D) by hydrogenating with PD/C to obtain the nitroalkane (E);
(ii) hydrogenating (F) with Pd/C in alcohol to obtain the amino-hydroxy acid (FA);
(iii) refluxing (FA) with p-toluenesulfonic acid and esterifying by refluxing in an alcohol to obtain (FF);
(iv) converting the ester to an amide by treating with NH₄OH and protecting the amino group of (FF) by treating with a base and a protecting group to obtain (G); and
(v) heating (G) in a solution of HCl in alcohol.

7. A process for preparing 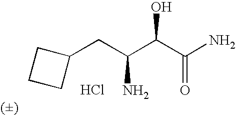
the process comprising:
(i) reacting (E) with glyoxylic acid and triethylamine, followed by benzylamine, to form a single diastereomer, F-BA benzylamine salt: 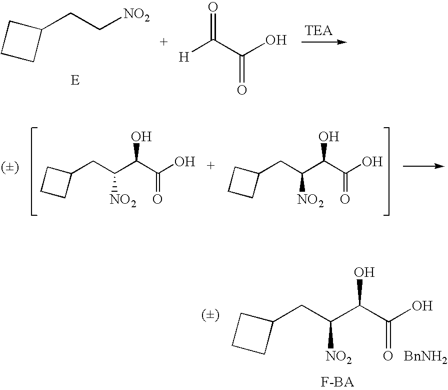
(ii) acidifying and reducing (F) to obtain the amine (FA), then esterifying and converting to the HCl salt of (FF): 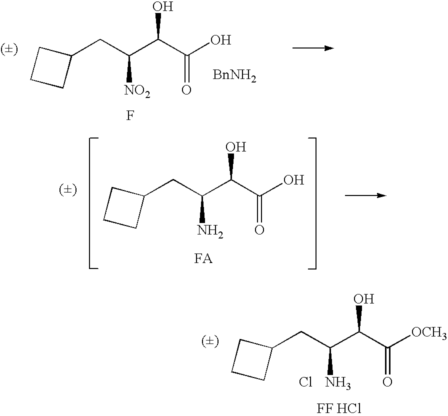
(iii) converting (FF-HCl) to the amide, deprotecting, and protecting the amino group to obtain (G); and 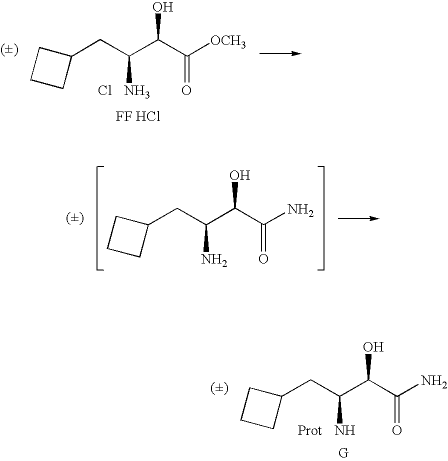
(iv) heating (G) in an alcohol solution of HCl.

8. A process for preparing 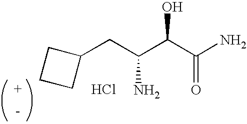
the process comprising:
(i) reacting (E) with glyoxylic acid and triethylamine, followed by dicyclohexylamine, to form a single diastereomer, F dicyclohexylamine salt: 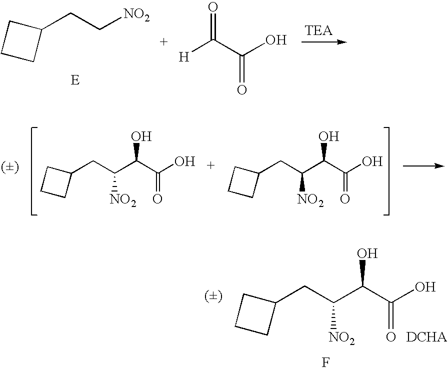
(ii) acidifying and reducing (F) to the amine (FA), then esterifying and converting to the HCl salt of (FF): 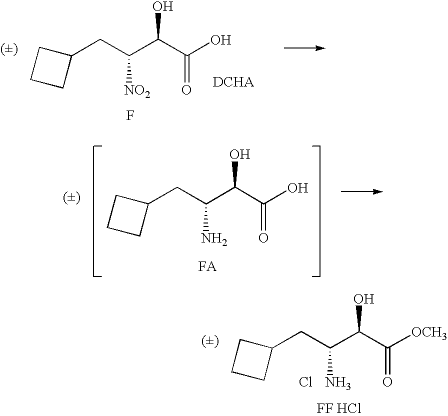
(iii) converting (FF HCl) to the amide, deprotecting, and protecting the amino group to obtain (G); and 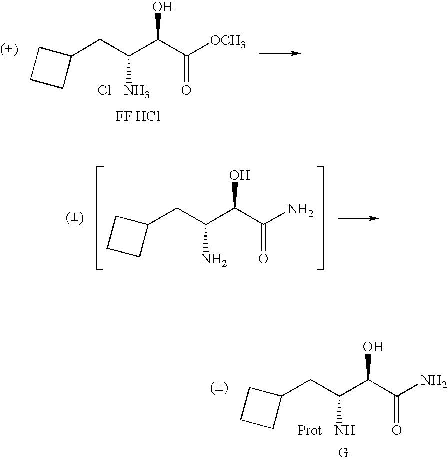
(iv) heating (G) in an alcohol solution of HCl.

9. The compound of any of the following formulae:

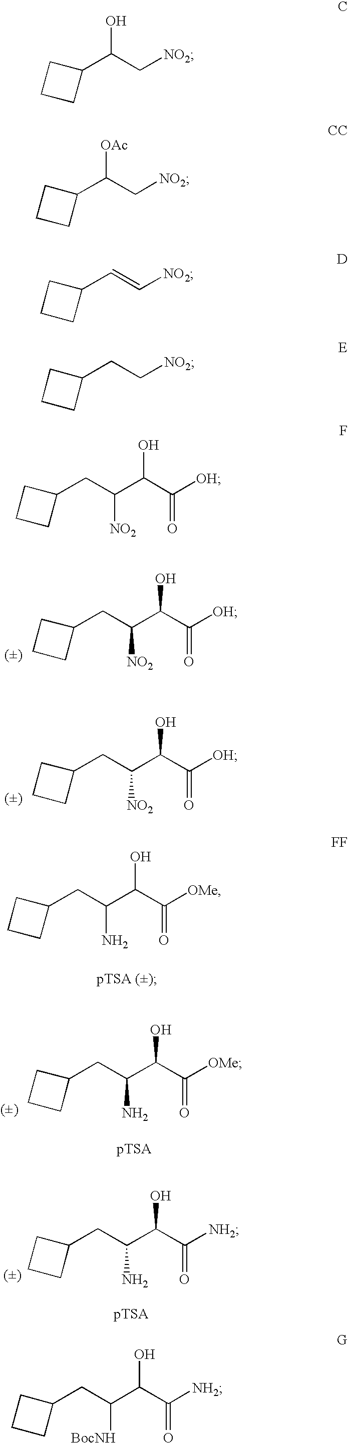

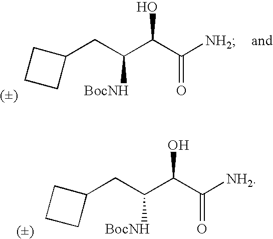

10. The process of claim 1 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.

11. The process of claim 10 wherein the ratio of diasteromers of compounds FF-HCl, G, and H produced after employing the precipitated compound F reflect the ratio of isomers precipitated for compound F.

12. The process of claim 2 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.

13. The process of claim 3 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.

14. The process of claim 4 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.

15. The process of claim 5 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DOHA is selected and from about 1:13 to about 1:20 when benzylamine is selected.

16. The process of claim 6 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.

17. The process of claim 7 wherein in compound F is obtained as an amine salt precipitate using an amine selected from dicyclohexylamine and benzylamine prior to carrying out the subsequent steps, said precipitate containing a ratio of major to minor isomers of from about 1:9 to about 1:14 when DCHA is selected and from about 1:13 to about 1:20 when benzylamine is selected.
